(19) United States
(12) Patent Application Publication
(10) Pub. No.: US 2017/0360435 A1
(43) Pub. Date: Dec. 21, 2017

(54) SURGICAL CLOSURE APPARATUS AND METHOD

(71) Applicant: Covidien LP, Mansfield, MA (US)

(72) Inventors: Inderjeet Singh Bhalla, Hyderabad (IN); Neeraj Kumar, Noida (IN)

(21) Appl. No.: 15/606,236

(22) Filed: May 26, 2017

Related U.S. Application Data
(60) Provisional application No. 62/352,045, filed on Jun. 20, 2016.

(51) Int. Cl. A61B 17/04; A61B 17/34; A61B 17/06; A61B 17/00

(52) U.S. Cl. CPC A61B 17/0482; A61B 17/0469; A61B 17/06066; A61B 17/34; A61B 17/3478; A61B 17/04; A61B 2017/0445; A61B 2017/047; A61B 2017/06052; A61B 2017/0608; A61B 2017/00637

(57) ABSTRACT

A surgical closure apparatus includes an outer member dimensioned for positioning within a wound opening, a needle assembly at least partially positioned within the outer member and a suture configured for at least partially closing the wound opening within the tissue. The needle assembly includes an elongate member defining a longitudinal opening, a suture needle coupled to the elongate member and extending to a needlepoint and being configured for movement between an unarmed condition where the needlepoint is in a relative radial inward position and an armed condition where the needlepoint is in a relative radial outward position and a deployment member extending at least partially through the longitudinal opening of the elongate member and coupled to the suture needle. The deployment member is movable within the longitudinal opening to permit the suture needle to move between the unarmed condition and the armed condition.

### CROSS-REFERENCE TO RELATED APPLICATIONS

This application claims the benefit of and priority to U.S. Provisional Patent Application No. 62/352,045 filed Jun. 20, 2016, the entire disclosure of which is incorporated by reference herein.

### BACKGROUND

### 1. Technical Field

The present disclosure relates to wound closure and, more particularly, relates to an apparatus and associated method for closing a wound or port opening in the abdomen in conjunction with a laparoscopic surgical procedure.

### 2. Background of Related Art

Puncture wounds may result from trauma or may be intentionally created to provide access to a body cavity during a surgical procedure. In an endoscopic or laparoscopic surgical procedure, for example, a trocar is utilized to puncture the abdomen to provide access by way of a cannula through the abdominal wall. Generally, the cannula or other access portal device is placed through the abdominal wall for introduction of surgical instrumentation required to perform the surgical procedure. Once the procedure is complete, it is necessary to close the puncture wound.

Current methods of wound closure are highly skill dependent as advancing a needle into a small port and ensuring that the needle crosses through different layers of abdominal tissue, including subcutaneous tissue, fascia and muscle, is quite cumbersome and difficult. In addition, there is a potential of the needle inadvertently contacting and injuring organs within the abdominal cavity.

### SUMMARY

Accordingly, the present disclosure is directed to further improvements in wound closure. In one embodiment, a surgical closure apparatus for facilitating closure of a wound includes an outer member dimensioned for positioning within a wound opening and defining a central longitudinal axis, a needle assembly at least partially positioned within the outer member, and a suture configured for at least partially closing the wound opening. The needle assembly includes an elongate member defining a longitudinal opening along a portion of a length thereof, a suture needle coupled to the elongate member and extending to a needlepoint and being configured for movement between an unarmed condition where the needlepoint is in a relative radial inward position and an armed condition where the needlepoint is in a relative radial outward position, and a deployment member extending at least partially through the longitudinal opening of the elongate member and coupled to the suture needle. The deployment member is movable within the longitudinal opening to permit the suture needle to transition between the unarmed condition and the armed condition. The suture is coupled to the suture needle and at least partially extends through the outer member.

In embodiments, the deployment member includes a flexible filament. In some embodiments, the deployment member is secured to the suture needle adjacent the needlepoint. In some aspects, the suture needle is normally biased toward the armed condition, and is configured to move to the unarmed condition in response to a tensile force exerted on the deployment member. In some embodiments, the needlepoint of the suture needle is disposed radial outward relative to the outer member when in the armed condition and is disposed radial inward relative to the outer member when in the unarmed condition. In some aspects, the elongate member defines an exit port in communication with the longitudinal opening where the deployment member extends through the exit port for securement to the suture needle.

In embodiments, the outer member defines a first longitudinal passage for reception of the elongate member of the needle assembly and may define a second longitudinal passage for accommodating the suture. In some embodiments, the suture needle is a curved suture needle. The suture needle may include an eye for receiving a portion of the suture.

A method for facilitating closure of a wound opening is also disclosed. The method includes:

positioning an outer member relative to a wound opening, the outer member having an elongate member at least partially disposed therein and a suture needle coupled to the elongate member and extending beyond the outer member, and terminating in a needlepoint;

securing a flexible deployment member at least partially extending within a longitudinal opening of the elongate member to the suture needle;

coupling a suture to the suture needle adjacent the needlepoint;

introducing the outer member within the wound opening while the suture needle is in an unarmed condition whereby the needlepoint is in a radial inward position relative to the outer member;

translating the deployment member within the longitudinal opening of the elongate member to transition the suture needle from the unarmed condition to an armed condition whereby the needlepoint is in a radial outward position relative to the outer member;

moving the elongate member within the wound opening through a first manipulation while the suture needle is in the armed condition such that the needlepoint and at least a portion of the suture needle penetrates first tissue portions surrounding the wound opening to cause a first suture segment of the suture to pass through the first tissue portions;

rearranging the outer member and the elongate member relative to the wound opening;

moving the elongate member within the wound opening through a second manipulation while the suture needle is in the armed condition such that the needlepoint and at least a portion of the suture needle penetrates second tissue portions surrounding the wound opening to cause a second suture segment of the suture to pass through the second tissue portions; and securing the first and second suture segments to at least partially close the wound opening.

In some embodiments, introducing the outer member includes applying a tensile force to the deployment member to at least partially bend the suture needle to move the suture needle to the unarmed condition. In embodiments, the suture needle is a curved suture needle and is normally biased toward the armed condition, and wherein translating the deployment member includes releasing the deployment member to permit the deployment member to advance within the longitudinal opening of the elongate member and allow the suture needle to assume the armed condition. In aspects, translating the deployment member includes extending the deployment member through a side exit port in the elongate member. In embodiments, introducing the outer member includes releasably securing the deployment member relative to the outer member to retain the suture needle in the unarmed condition.

In some embodiments, the method includes positioning the elongate member within a first longitudinal passage of the outer member and extending the suture through a second longitudinal passage of the outer member.

In embodiments, the wound opening extends through the abdominal cavity and wherein moving the elongate member through each of the first and second manipulations includes advancing the first and second suture segments through at least fascia tissue surrounding the abdominal cavity.

The surgical closure apparatus and method of use provides an effective and safe approach for closing a wound or port opening in tissue, particularly, within the abdominal cavity. The movement of the needle assembly between the unarmed and armed conditions of the suture needle is directly controlled by the clinician. The incorporation of the needle assembly within the apparatus removes the necessity of individually introducing one or more needles through an access port to suture the surrounding wound, which is a highly skill dependent surgical task and may introduce additional trauma and incur additional recovery time.

Other advantages of the present disclosure will be appreciated from the following description.

### BRIEF DESCRIPTION OF THE DRAWING(S)

Embodiments of the present disclosure will be appreciated by reference to the accompanying drawings wherein:

FIGS. 1-2 are first and second perspective views of the surgical closure apparatus in accordance with the present disclosure illustrating the outer member, the needle assembly at least partially disposed within the outer member, and the suture for closing a wound opening;

FIG. 3 is an exploded perspective view of the surgical closure apparatus illustrating the elongate member, the suture needle and the deployment member of the needle assembly, and the outer member;

FIG. 4 is a partial side cross-sectional view of the surgical closure apparatus of FIG. 1 illustrating the needle assembly in an armed condition through release of the deployment member coupled to the suture needle;

FIG. 5 is a partial side cross-sectional view of the surgical closure apparatus illustrating the suture needle of the needle assembly in an unarmed condition through application of a tensile force on the deployment member; and

FIGS. 6A-6F illustrate a sequence of use of the surgical closure apparatus in closing a wound opening within the abdomen.

### DETAILED DESCRIPTION

Particular embodiments of the present disclosure are described hereinbelow with reference to the accompanying drawings; however, it is to be understood that the disclosed embodiments are merely examples of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to employ the present disclosure in virtually any appropriately detailed structure.

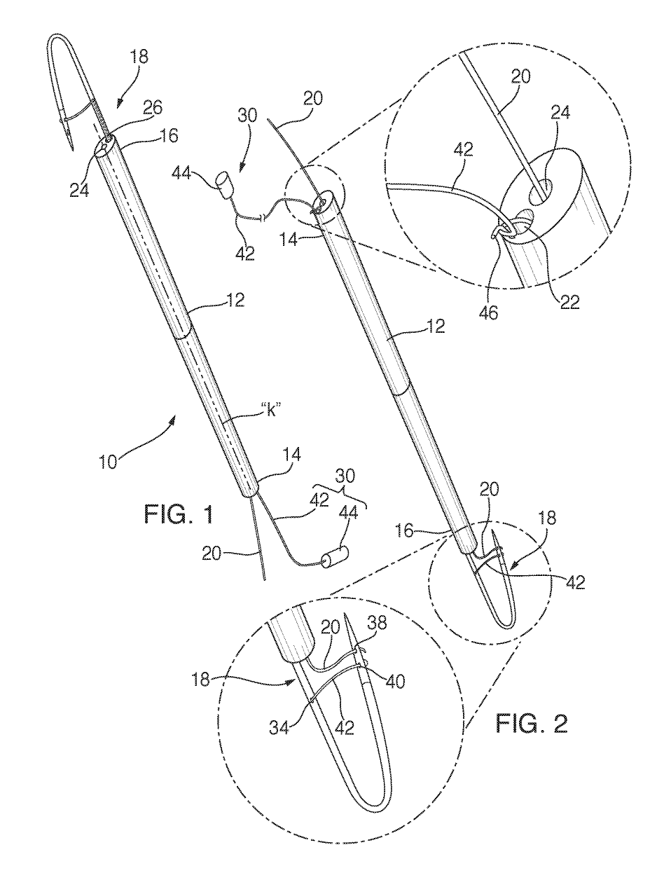

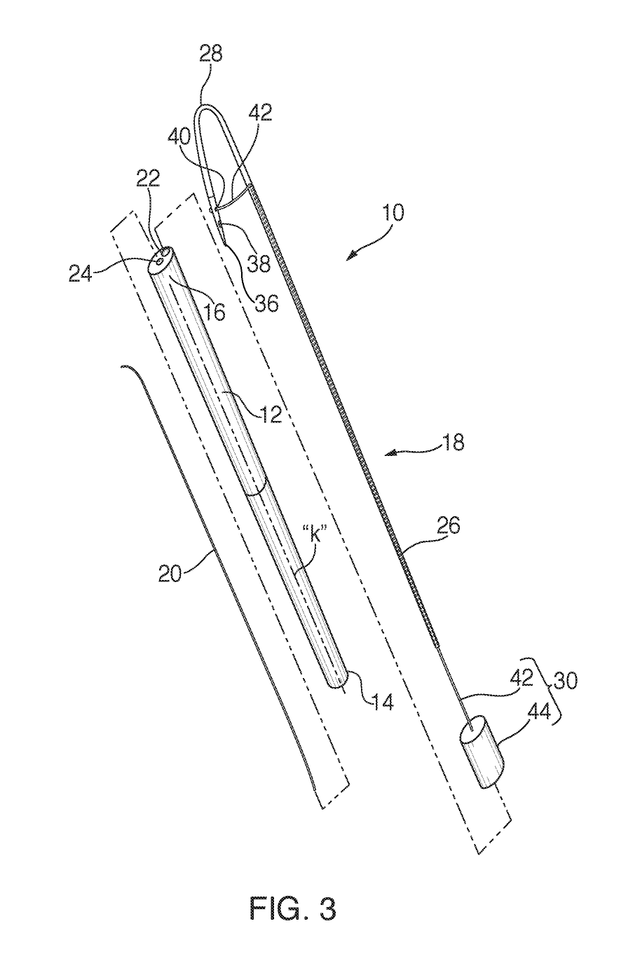

Referring now to the drawings where like reference numerals indicate similar components throughout the several views, FIGS. 1-4 illustrate the surgical closure apparatus 10 of the present disclosure. The surgical closure apparatus 10 is adapted to facilitate the closure of a wound opening in tissue, and, has particular application in the closure of a puncture or port wound created within the abdomen, e.g., through the abdominal wall, in connection with a laparoscopic surgical procedure. However, the surgical closure apparatus 10 and associated method of use may be used to close a wound opening in other areas of the subject's body whether created during a surgical procedure or resulting from accident or trauma.

The surgical closure apparatus 10 includes an elongated outer member 12 defining proximal and distal end portions 14, 16, a needle assembly 18 at least partially positionable within the outer member 12 and a suture 20 coupled to the needle assembly 18. The outer member 12 may be monolithically formed, or alternatively, include several components secured to each other via conventional means. The outer member 12 defines a central longitudinal axis “k”, and has first and second longitudinal passages 22, 24. The first longitudinal passage 22 is radially spaced relative to the central longitudinal axis “k” and the second longitudinal passage 24 is in general longitudinal alignment with the central longitudinal axis “k”. Other arrangements are also envisioned. The outer member 12 may be solid, in whole or in part, with the exception of the first and second longitudinal passages 22, 24, or alternatively, may be hollow and include internal tubes which define the first and second longitudinal passages 22, 24. The outer member 12 may be generally cylindrical along a majority of its length although other dimensions are also envisioned.

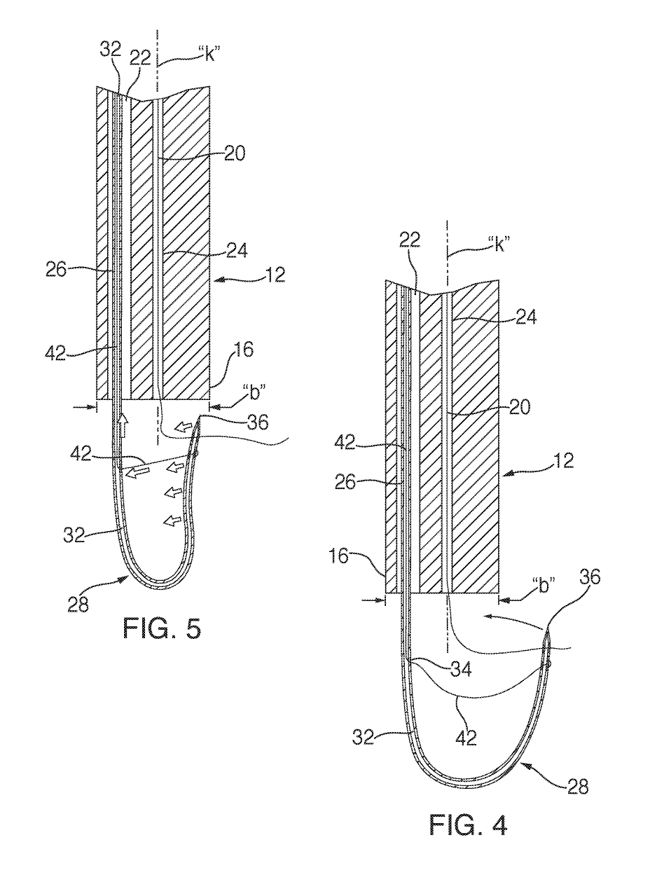

With continued reference to FIGS. 1-4, the needle assembly 18 includes an elongate member 26, a suture needle 28 connected to the elongate member 26 and a deployment member 30 at least partially extending within the elongate member 26. The elongate member 26 is at least partially, e.g., fully disposed, within the first longitudinal passage 22, and may be secured within the first longitudinal passage 22 through conventional means. In the alternative, the elongate member 26 may move in a longitudinal direction within the first longitudinal passage 22. The elongate member 26 of the needle assembly 18 defines a longitudinal opening 32 (FIG. 4) along at least a portion of its length for accommodating the deployment member 30. In one embodiment, the longitudinal opening 32 extends through the elongate member 26 and through at least a portion of the suture needle 28, e.g., the elongate member 26 and the suture needle 28 are hollow. The elongate member 26 defines an exit port 34 in communication with the longitudinal opening 32 adjacent the distal end portion 16.

The suture needle 28 is disposed beyond the distal end portion 16 of the outer member 12. The suture needle 28 is a curved needle, e.g., generally J-shaped, which leads to a needlepoint 36. In the alternative, the suture needle 28 may be linear and offset relative to the longitudinal axis “k” such that the needlepoint 36 extends toward the housing, e.g., generally V-shaped. The suture needle 28 may be monolithically formed with the elongate member 26 or a separate component secured to the elongate member 26 through conventional means. The suture needle 28 is fabricated from a relative rigid material including stainless steel, spring steel, shape memory alloys such as Nitinol and other metals or polymeric materials, but has sufficiently flexibility to be at least partially bent during use of the apparatus 10. The suture needle 28 may include an eye 38 extending through the body of the needle 28 for reception and passage of the suture 20 and a second opening 40 for securement of the deployment member (FIG. 2).

The deployment member 30 includes a flexible filament 42 and, optionally, a handle 44 (FIGS. 1 and 2) which is coupled to the flexible filament 42 and dimensioned for engagement by the clinician. The flexible filament 42 has sufficient tensile strength to bend the suture needle 28 upon application of a tensile force to the flexible filament 42. Suitable materials for the flexible filament 42 of the deployment member 30 include polymeric materials, nylon, steel or the like. The flexible filament 42 extends through the longitudinal opening 32 of the elongate member 26 and extends outwardly from the proximal end portion 14 of the outer member 12 where it is coupled to the handle 44. The flexible filament 42 also extends through the exit port 34 of the elongate member 26 for securement to the suture needle 28. In one embodiment, the flexible filament 42 extends through the second opening 40 in the suture needle 28 adjacent the needlepoint 36, and is secured within the second opening 40 through conventional means including knotting, adhesives or the like. Alternatively, the flexible filament 42 may be welded, secured and/or adhered directly to the outer surface of the suture needle 28.

With continued reference to FIGS. 1-4, the suture 20 extends through the eye 38 of the suture needle 28 and through the second longitudinal passage 24 of the outer member 12 external of the proximal end portion 14 of the outer member 12. In one embodiment, the suture 20 is not secured relative to the suture needle 28 and freely passes through the eye 38. In the alternative, the suture 20 may be secured relative to the eye 38. The suture 20 is capable of advancing or sliding through the second longitudinal passage 24 during use of the apparatus 10 in closing the wound opening. Suitable materials of fabrication for the suture 20 include natural or synthetic degradable materials, non-degradable materials, or combinations thereof.

FIG. 4 illustrates the armed condition of the suture needle 28. In the armed condition, the needlepoint 36 and, possibly, at least a portion of the suture needle 28 extending from the needle point 36 are disposed radially outward relative to the outer boundary “b” or external diameter of the outer member 12, and positioned to engage the tissue surrounding the wound opening. The suture needle 28 is normally biased to the armed condition, i.e., it is the normal shape of the suture needle 28.

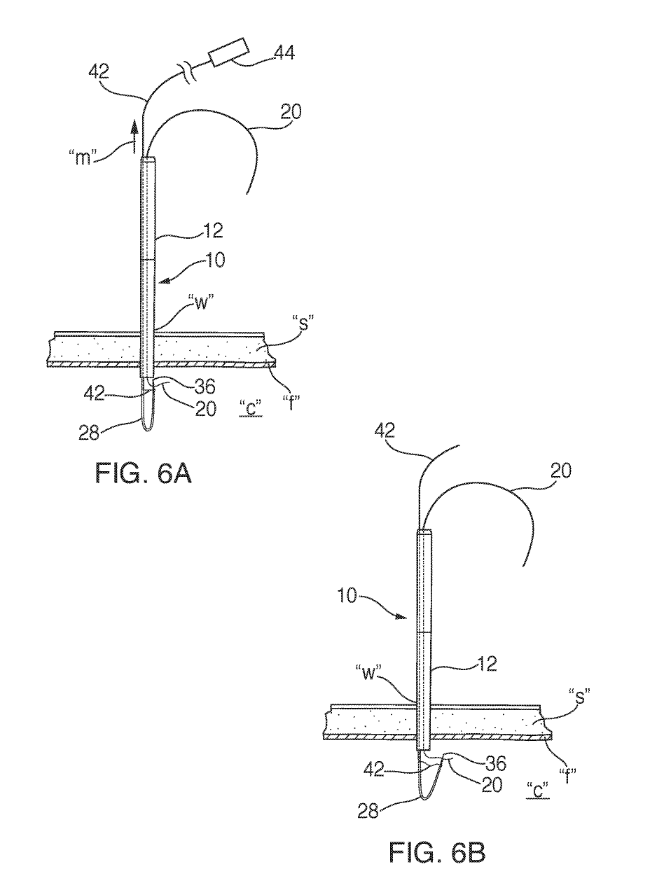

FIG. 5 illustrates the unarmed condition of the suture needle 28. The unarmed condition is effected by pulling or retracting the flexible filament 42 of the deployment member 30 in a proximal direction toward the clinician through engagement with, e.g., the handle 44, to apply a tensile force to the flexible filament 42 which is conveyed to the suture needle 28. When subjected to this force, the suture needle 28 bends toward the elongate member 26 or radially inwardly relative to the central longitudinal axis ‘k”. In the unarmed condition, the suture needle 28 is disposed radially inward relative to the central longitudinal axis “k” such that the suture needle 28 and the needlepoint 36 are generally confined within the outer boundary or diameter “b” defined by the outer member 12. In one embodiment, the outer member 12 includes a catch or hook 46 (FIG. 2) adjacent the proximal end portion 14 about, or through, which the flexible filament 42 is positioned to securely retain the flexible filament 42 in a tensioned state and the suture needle 28 in the unarmed condition. Other arrangements for releasably securing the flexible filament 42 in the tensioned state may include a compressible ferrule on the flexible filament 42, which is slidable to be at least partially received within the first longitudinal passage 22. In this manner, the ferrule would be compressed about the flexible filament 42 to secure the filament 42, and also be secured with the outer member 12 through engagement with the portions of the outer member 12 defining the first longitudinal passage 22.

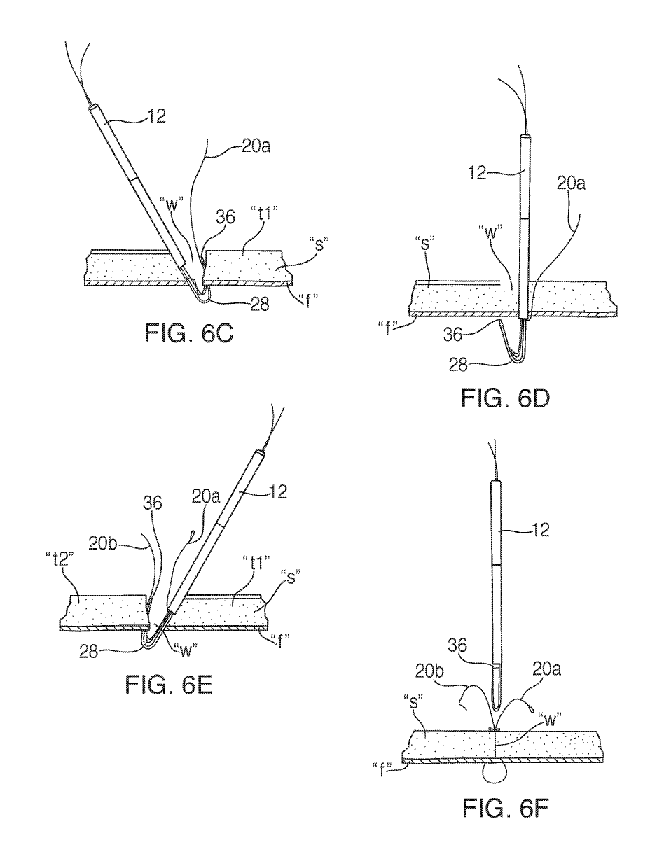

FIGS. 6A-6F illustrate a method of use of the closure apparatus 10 in closing a wound opening. The following discussion will focus on the use of the apparatus in closing a puncture or port wound created by an obturator or trocar during a laparoscopic procedure. However, it is envisioned that the apparatus 10 may have application in closure of wounds due to trauma in any area of the body.

Subsequent to the performance of a laparoscopic procedure or maneuver within the abdominal cavity, the attention of the clinician is directed to closing the puncture or port wound extending through the abdominal cavity. The clinician grasps the apparatus 10 and threads the suture 20 through the second longitudinal passage 24 and through the eye 38 of the suture needle 28. In one method, the suture 20 is not secured relative to the eye 38. The needle assembly 18 and suture needle 28 are placed in the unarmed condition of FIG. 6A by pulling on the flexible filament 42 of the deployment member 30 in the direction of directional arrow “m” through engagement with the handle 44, which places the flexible filament 42 under tension and bends the suture needle 28 and the needlepoint 36 radially inwardly relative to the central longitudinal axis “k” (see also FIG. 5). The flexible filament 42 may be secured to the catch 46 adjacent the proximal end portion 14 of the outer member 12 (FIG. 2) to releasably retain the flexible filament 42 and, thus, the suture needle 28 in the unarmed condition.

In the unarmed condition, the apparatus 10 is then introduced or positioned within the wound opening “w” extending through the subcutaneous tissue “s”, fascia “f”, muscle and abdominal lining of the abdominal wall. The apparatus 10 is advanced such that at least the suture needle 28 enters the abdominal cavity “c”.

Once the suture needle 28 is disposed within the abdominal cavity “c”, the suture needle 28 is moved to the armed condition by releasing the flexible filament 42 from the catch 46 and permitting the flexible filament 42 to advance within the longitudinal opening 32 of the elongate member 26 with at least a portion of the flexible filament 42 exiting through the exit port 34 in response to the bias of the suture needle 28 to its outward armed condition. Specifically, the suture needle 28, which is no longer subjected to the forces of the flexible filament 42, assumes its normal armed condition depicted in FIG. 6B. In the armed condition, at least the needlepoint 36 of the suture needle 28 is disposed outwardly of the outer member 12 with the suture 20 coupled to the eye 38 of the suture needle 28 and extending back through the second longitudinal passage 24. Thereafter, the apparatus 10, including the outer member 12 and/or the needle assembly 18, is at least partially withdrawn relative to the wound opening “w”, e.g., pulled in a proximal direction toward the clinician, which causes the needlepoint 36 and the suture needle 28 to pierce through first tissue portions “t1”, including fascia “f”, abdominal muscle and/or subcutaneous tissue “s”, to thereby pass a first suture segment 20a of the suture 20 through the first tissue portions “t1”, as depicted in FIG. 6C. The apparatus 10 is retracted a distance to expose the first suture segment 20a of the suture 20 to the clinician. The first suture segment 20a may be grasped with, e.g., forceps, to pull the first suture segment 20a through the tissue outwardly of the abdominal cavity “c”. During this movement of the first suture segment 20a, the suture 20 advances or slides through the second longitudinal passage 24 of the outer member 12. The apparatus 10 is then advanced back within the wound opening “w” while the first suture segment 20a is retained or secured external of the abdominal cavity “c”. As the apparatus 10 is advanced within the wound opening “w”, the suture 20 slides within the second longitudinal passage 24 of the outer member 12.

Referring now to FIG. 6D, the entire apparatus 10 is reoriented or rotated within the wound opening “w” through an arc segment of, e.g., 180°, to arrange the suture needle 28 adjacent the opposed tissue on the other side of the wound opening “w”. The apparatus 10 is again at least partially withdrawn relative to the wound opening “w” or pulled proximally toward the clinician as depicted in FIG. 6E which causes the needlepoint 36, the suture needle 28 and attached suture 20 to pass through the second tissue portions “t2” surrounding the wound opening “w”, including fascia “f” and/or the subcutaneous tissue “s”, to expose the second suture segment 20b of the suture 20 from the abdominal cavity “c”. During this movement, the suture 20 slides through the second longitudinal passage 24 of the outer member 12 in a similar manner as discussed hereinabove. With the first and second suture segments 20a, 20b exposed from the abdominal cavity “c” and passing through respective first and second tissue portions “t1”, “t2” surrounding the wound opening “w”, the remaining slack in the suture 20 is removed by pulling on the second suture segments 20b which causes the suture 20 to translate through the second longitudinal passage 24 and be released from the suture needle 28 and the outer member 12. The apparatus 10 is removed, and the suture 20 is tightened with the first and second suture segments 20a, 20b tied off in a conventional manner to close the wound opening “w” as shown in FIG. 6F.

Although the above-described method of wound closure includes passing two segments through tissue surrounding the wound opening “w” to close the wound, it is envisioned that more than two suture segments may be utilized. For example, four suture segments may be applied around the wound opening, which would entail, e.g., rotating the apparatus 10 through an arc sector of approximately 90° within the wound opening subsequent to application of each suture segment.

The above description and the drawings are provided for the purpose of describing embodiments of the present disclosure and are not intended to limit the scope of the disclosure in any way. It will be apparent to those skilled in the art that various modifications and variations can be made without departing from the spirit or scope of the disclosure. Thus, it is intended that the present disclosure cover the modifications and variations of this disclosure provided they come within the scope of the appended claims and their equivalents.

What is claimed is:

1. A surgical closure apparatus for facilitating closure of a wound, comprising:
an outer member dimensioned for positioning within a wound opening and defining a central longitudinal axis;
a needle assembly at least partially positioned within the outer member, the needle assembly including:
an elongate member defining a longitudinal opening along a portion of a length thereof;
a suture needle coupled to the elongate member and extending to a needlepoint, the suture needle configured for movement between an unarmed condition where the needlepoint is in a relative radial inward position and an armed condition where the needlepoint is in a relative radial outward position; and
a deployment member extending at least partially through the longitudinal opening of the elongate member and coupled to the suture needle, the deployment member movable within the longitudinal opening to permit the suture needle to move between the unarmed condition and the armed condition; and
a suture configured for at least partially closing the wound opening, the suture coupled to the suture needle and at least partially extending through the outer member.

2. The surgical closure apparatus according to claim 1 wherein the deployment member includes a flexible filament.

3. The surgical closure apparatus according to claim 2 wherein the deployment member is secured to the suture needle adjacent the needlepoint.

4. The surgical closure apparatus according to claim 3 wherein the suture needle is normally biased toward the armed condition, and is configured to move to the unarmed condition in response to a tensile force exerted on the deployment member.

5. The surgical closure apparatus according to claim 4 wherein the needlepoint is disposed radial outward relative to the outer member when in the armed condition and is disposed radial inward relative to the outer member when in the unarmed condition.

6. The surgical closure apparatus according to claim 5 wherein the elongate member defines an exit port in communication with the longitudinal opening, the deployment member extending through the exit port for securement to the suture needle.

7. The surgical closure apparatus according to claim 5 wherein the outer member defines a first longitudinal passage for reception of the elongate member of the needle assembly.

8. The surgical closure apparatus according to claim 7 wherein the outer member defines a second longitudinal passage for accommodating the suture.

9. The surgical closure apparatus according to claim 5 wherein the suture needle includes an eye for receiving a portion of the suture.

10. The surgical closure apparatus according to claim 5 wherein the suture needle is a curved suture needle.

11. A method for facilitating closure of a wound opening, comprising:
positioning an outer member relative to a wound opening, the outer member having an elongate member at least partially disposed therein and a suture needle coupled to the elongate member extending beyond the outer member and terminating in a needlepoint;
securing a flexible deployment member at least partially extending within a longitudinal opening of the elongate member to the suture needle;
coupling a suture to the suture needle adjacent the needlepoint;
introducing the outer member within the wound opening while the suture needle is in an unarmed condition whereby the needlepoint is in a radial inward position relative to the outer member;
translating the deployment member within the longitudinal opening of the elongate member to transition the suture needle from the unarmed condition to an armed condition whereby the needlepoint is in a radial outward position relative to the outer member;
moving the elongate member within the wound opening through a first manipulation while the suture needle is in the armed condition such that the needlepoint and at least a portion of the suture needle penetrates first tissue portions surrounding the wound opening to cause a first suture segment of the suture to pass through the first tissue portions;
rearranging the outer member and the elongate member relative to the wound opening;
moving the elongate member within the wound opening through a second manipulation while the suture needle is in the armed condition such that the needlepoint and at least a portion of the suture needle penetrates second tissue portions surrounding the wound opening to cause a second suture segment of the suture to pass through the second tissue portions; and
securing the first and second suture segments to at least partially close the wound opening.

12. The method according to claim 11 wherein the suture needle is a curved suture needle and is normally biased toward the armed condition, and wherein translating the deployment member includes releasing the deployment member to permit the deployment member to advance within the longitudinal opening of the elongate member and allow the suture needle to assume the armed condition.

13. The method according to claim 12 wherein introducing the outer member includes applying a tensile force to the deployment member to at least partially bend the suture needle to move the suture needle to the unarmed condition.

14. The method according to claim 13 wherein translating the deployment member includes extending the deployment member through a side exit port in the elongate member.

15. The method according to claim 11 wherein introducing the outer member includes releasably securing the deployment member relative to the outer member to retain the suture needle in the unarmed condition.

16. The method according to claim 11 including positioning the elongate member within a first longitudinal passage of the outer member.

17. The method according to claim 16 including extending the suture through a second longitudinal passage of the outer member.

18. The method according to claim 11 wherein the wound opening extends through the abdominal cavity and wherein moving the elongate member through each of the first and second manipulations includes advancing the first and second suture segments through at least fascia tissue surrounding the abdominal cavity.
